(19) United States
(12) Patent Application Publication
(10) Pub. No.: US 2005/0267290 A1
(43) Pub. Date: Dec. 1, 2005

(54) Preparation of acid amides

(75) Inventors: Max Braun, Wedemark (DE); Kerstin Eichholz, Langenhagen (DE)

Correspondence Address: CROWELL & MORING LLP;INTELLECTUAL PROPERTY GROUP, P.O. BOX 14300, WASHINGTON, DC, 20044-4300, US

(73) Assignee: Solvay Fluor und Derivate GmbH, Hannover (DE)

(21) Appl. No.: 10/944,418

(22) Filed: Sep. 20, 2004

Related U.S. Application Data
(63) Continuation of application No. PCT/EP03/02453, filed on Mar. 11, 2003.

(30) Foreign Application Priority Data
Mar. 21, 2002 (DE) DE 102 12 526.0

(51) Int. Cl. C07K 14/00

(52) U.S. Cl. 530/300; 530/402

(57) ABSTRACT

A method and apparatus suitable, inter alia, for producing carboxylic acid amides from carboxylic acid esters and amines in which a mixture of amide and alcohol is obtained, the mixture is maintained in a liquid state, and the alcohol which is released is progressively evaporated, after which the liquid carboxylic acid freed from alcohol contamination is discharged from the reactor, divided into small portions, and the small portions are converted by cooling into solid particles. The method and apparatus can also be used to prepare peptides and other solid organic compounds, generally.

### CROSS REFERENCE TO RELATED APPLICATIONS

This application is continuation of international patent application no. PCT/EP03/02453, filed Mar. 11, 2003, designating the United States of America, and published in German on Oct. 2, 2003 as WO 03/080563, the entire disclosure of which is incorporated herein by reference. Priority is claimed based on Federal Republic of Germany patent application no. DE 102 12 526.0, filed Mar. 21, 2002.

### BACKGROUND OF THE INVENTION

The present invention relates to a method for the preparation of solidified organic compounds, especially of acid amides, the carboxylic acid portion of which is substituted by at least one fluorine atom.

Perfluorinated acid amides are intermediates in chemical syntheses. Trifluoroacetamide, for example, may be used as a reagent for the preparation of primary amines from halides or mesylates. N-methyltrifluoroacetamide is an intermediate for the preparation of N-silylated derivatives. Moreover, it may be used as a reagent for the preparation of secondary N-methylalkylamines. Other partially fluorinated amides may be converted into corresponding unsaturated amides or into nitrites (see, e.g., U.S. Pat. No. 2,730,543). The resulting compounds are then starting materials for the synthesis of polymers and copolymers. Partially fluorinated amines can be obtained by hydrogenating corresponding fluorinated amides and also corresponding N-substituted or N,N-substituted amides.

In accordance with a simple method of preparation, esters of fluorinated carboxylic acids are reacted with the appropriate amine. In so doing, alcohol is formed. The amide precipitates and, in accordance with a simple method of isolation, the precipitated amide is filtered out and then dried. Such a method has disadvantages when employed for large scale industrial applications, because the filters become blocked and the product is contaminated by encapsulated alcohol.

### SUMMARY OF THE INVENTION

It is an object of the present invention to provide an improved method of preparing perfluorinated carboxylic acid amides.

Another object is to provide a method of preparing solid compounds, such as carboxylic acid amides, which reduce contamination of the desired product.

A further object of the invention is to provide a method of preparing solid compounds such as carboxylic acid amides which is less susceptible to filter blockage.

An additional object of the invention is to provide an apparatus which enables solid organic compounds in general, and acid amides in particular, to be prepared and isolated in a simple manner.

These and other objects are achieved in accordance with the present invention by providing a method of preparing an organic compound which has a melting point higher than 30° C. at ambient pressure and is admixed with at least one organic contaminant having a boiling point of up to 120° C. comprising heating the mixture so that the mixture is maintained in a liquid state and the at least one organic contaminant is evaporated, dividing the liquid from which the at least one organic contaminant has been evaporated into small portions, and cooling the small portions to convert the portions into solid particles, in which the organic compound is thermally stable up to the boiling point thereof, and the boiling point of the organic compound is at least 40° C. higher than the boiling point of the at least one contaminant.

In accordance with a further aspect of the invention, the objects are achived by providing an apparatus for producing a solid compound freed of a more volatile contaminant comprising a heated reactor, a rotatable perforated roller, a pipeline connecting the heated reactor to the perforated roller, and a cooled moving belt disposed to receive liquid droplets from said perforated roller.

The invention in its widest sense relates to a method for preparing an organic compound which has a melting point higher than 30° C. at ambient pressure and is present as a mixture with one or more organic contaminants, which in turn have boiling points of up to 120° C. The mixture is heated and kept in the liquid state, so that the organic contaminants evaporate. Then the organic compound, which has been freed from the evaporated organic impurities, is divided into smaller volumes, which are converted by cooling into the solid state. In accordance with the invention the organic compound is thermally stable up to its boiling point, and the boiling point of the organic compound is at least 40° C. higher than the boiling point of the contaminant(s).

Preferably, the organic compound is a peptide. Particularly preferably, the peptide is substituted in the chain by fluorine or has a fluorine-substituted protective group.

The method of the invention is especially suitable for the preparation of carboxylic acid amides corresponding to formula (I)
R—C(O)—NR¹R² (I)
in which
R represents C1 to C4 alkyl, which is substituted by at least one fluorine atom,
R¹ and R² are the same or different and represent hydrogen or C1 to C4 alkyl,
   or in which
R¹ and R² together with the nitrogen atom form a five-membered or six-membered ring system, the melting point of the compounds of formula (I) being at least 40° at standard pressure.
   In the method of the invention, an ester corresponding to formula (II)
   R—C(O)—OR³ (II)
   in which
R has the meaning given above, and
R³ represents C1 to C6 alkyl, substituted C1 to C6 alkyl or aryl, such as phenyl or benzyl,
   is reacted with an amine corresponding to formula (III),
   HNR¹R² (III)
   in which
R¹ and R² have the meaning given above.

The reaction is carried out in reactor, and a mixture of acid amides of formula (I) and the alcohol R³0H, which is set free, is thereby formed. In the method according to the invention, the alcohol R³OH is distilled off while the contents of the reactor are kept in the liquid state, and the liquid carboxylic acid amide of formula (I) is thereafter removed from the reactor and divided into smaller volumes, which are cooled to convert them to the solid-state.

The term “C1 to C4 alkyl” is also intended to include substituted C1 to C4 alkyl, such as C2 to C4 alkyl, which is substituted in the β position by a thiomethacrylate group, as discussed in U.S. Pat. No. 3,445,491.

R preferably represents CF₃, CF₂H, CF₂Cl, CF₃CFH or CF₃CF₂.

R¹ and R² may be identical or different and preferably represent hydrogen, CH₃, C₂H₅ or C₃H₇. Particularly preferably, R¹ and R² represent hydrogen, or R¹ represents hydrogen, and R² represents CH₃, C₂H₅ or C₃H₇.

R³ preferably represents CH₃, C₂H₅, C₃H₇ or CF₃CH₂.

The contents of the reactor are divided into “smaller volumes” so that the resulting small portions can be converted into the solid state within an acceptable period of time. Moreover, it is desirable that the size of the “smaller volumes” be selected so that particle sizes are attained which can be handled well technically. Preferably, the concept of “smaller volumes” denotes particles with a volume of 0.1 ml to 20 ml. However, particles having a larger or smaller volume can also be produced within the broad scope of the invention.

In accordance with a preferred embodiment, the acid amide of formula (I), divided into smaller volumes, is transferred to a cooled support, for example, by spraying or splashing. Preferably, it is allowed to drip onto the support, especially under the action of gravity. It is particularly preferred to use a cooled moving belt as cooled support. This cooled belt preferably is an endless belt. Water, for example, may be used as cooling medium.

The deposition of “smaller volumes” on the moving belt preferably is accomplished using a rotating, perforated roller. The carboxylic acid amide, which is maintained in the liquid state, is transferred into the roller and drips through the holes onto the cooled moving belt. The drops solidify and may then be scraped off. The alcohol preferably is evaporated under vacuum.

As noted above, the invention also relates to an apparatus for carrying out the method of the invention. The apparatus of the invention includes a reactor, which can be heated and is connected preferably over heated pipelines with a rotating, perforated roller. The apparatus furthermore comprises a cooled moving belt, preferably a cooled endless belt. The perforated roller advantageously is disposed so that liquids transferred to it, can drip onto the cooled moving belt under the action of gravity.

### BRIEF DESCRIPTION OF THE DRAWINGS

The invention will be described in further detail hereinafter with reference to an illustrative apparatus shown in the accompanying drawing, which is a schematic representation of an apparatus for carrying out the method of the invention.

### DETAILED DESCRIPTION OF PREFERRED EMBODIMENTS

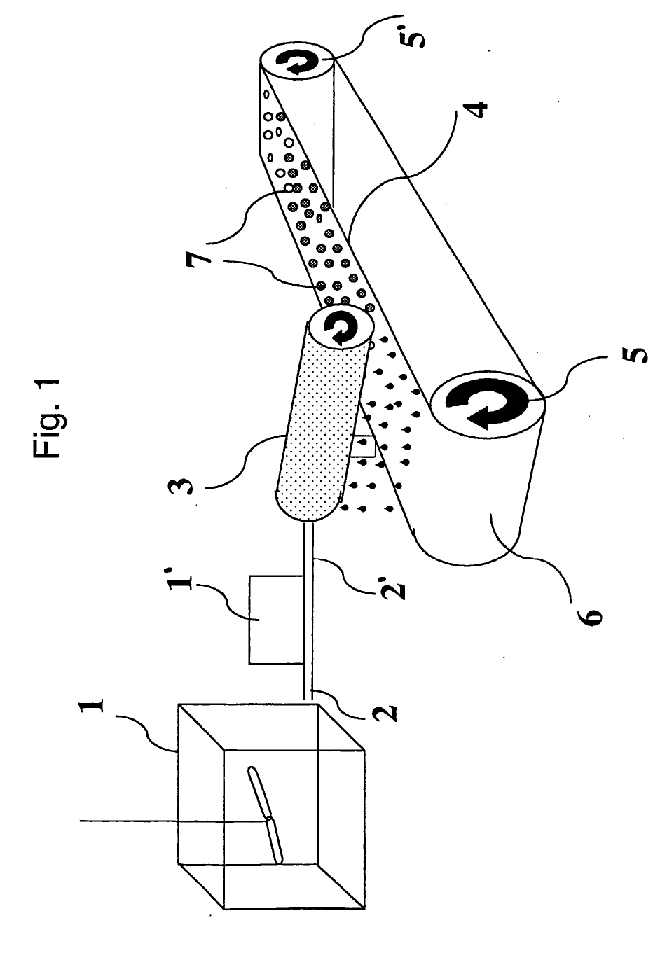

FIG. 1 shows a simple apparatus according to the invention for carrying out the method of the invention. The schematically illustrated reactor 1 is provided with a stirrer. For simplicity of illustration, pipelines for supplying reactants, as well as apparatus for producing a vacuum, measuring devices, etc., have been omitted from the drawing. The reactor is connected via a heated pipeline 2 to a distillate receiver 1′, which in turn is connected via a heated pipeline 2′ to a heated perforated roller 3. The liquid material, shown as drops 6, is transferred from the rotating perforated roller 3 to the endless moving belt 4. The endless moving belt is moved by driving rollers 5 and 5′. For simplicity of illustration, means for cooling the moving belt have been omitted from the drawing. However, the cooling may be effected, for example by spraying the underside of the moving belt with cold water. The drops 6 solidify on the cooled moving belt 4 and form solid particles 7. The particles 7 in turn fall from the moving belt at the turning point of the drive roller 5′ into a collecting container, which is provided. Optionally, a scraper may also be provided, with which the solid particles 7 are lifted from the moving belt and transferred to the container.

The invention enables solid compounds such as carboxylic acid amides to be produced easily on a large, industrial scale.

The following examples are intended to illustrate the invention in further detail without limiting its scope.

### EXAMPLE 1

### Preparation of N-methyltrifluoroacetamide from ethyl trifluoroacetate and methylamine using the cooled belt technology

Apparatus:

The reactor is a 90 liter Pfaudler vessel, which can be evacuated and is equipped with a stirrer, gas inlet and column connections. The gas is introduced through an immersion tube and a condenser. The reactor, or Pfaudler vessel, is connected by a heated pipeline to a receiver. The receiver is provided with the intention of avoiding pressure fluctuations. The receiver is connected via a further heated pipeline to a rotating perforated roller or rotoformer.

The reaction product, which leaves the rotoformer in the form of droplets, is transferred to a cooling belt. The cooling belt is comprised of a 150 mm wide endless, welded stainless steel belt, which is mounted on two driving rollers having a diameter of 150 mm. The rear driving roller is rotated by a motor through a chain drive. Nozzles are mounted beneath the cooling belt and spray a cooling medium directly onto the underside of the cooling belt. The length of the belt from the delivery unit (rotoformer) to the discharge point is 2.70 meters. At the end of the cooling belt, there is a scraper, which lifts the particle-shaped solid material from the belt.

The delivery unit, i.e., the rotating perforated roller or rotoformer, for the reaction product, which is maintained in liquid state, comprises a rotating roller with 15 holes per row. The holes have a diameter of 1.2 mm and are spaced a distance of 8 mm from one another along the row. The distance between the rows is also 8 mm. The holes of the individual rows are offset relative to one another. The rotoformer is also controlled via a DC motor and a chain drive.

Reaction:
CF₃CO₂C₂H₅+CH₃NH₂→CH₃CONHCH₃+C₂H₅OH
Formulation:
458.2 moles (65.1 kg) of ethyl trifluoroacetate
468.0 moles (15.0 kg) of methylamine

The ethyl trifluoroacetate was introduced into the reactor, and methylamine was then introduced at room temperature. As a result of the exothermic reaction, the temperature of the reaction mixture increased to 80° C. At the end of the reaction, the ethyl alcohol was distilled off under vacuum (20 mbar, starting temperature in the sump: 55° C.). After distillation was complete, the contents of the reactor were cooled to 60° C., the reactor was vented, and the reaction mixture was transferred to the receiver using a metering pump. The temperature of the receiver and the perforated roller was adjusted to 55° C. The moving belt was cooled with water at 13° C. The desired amide product was produced in the form of flakes at the rate of 13.7 kg per hour.

### EXAMPLE 2

### Preparation of trifluoroacetamide from ethyl trifluoroacetate and ammonia

Reaction:
CF₃CO₂C₂H₅+NH₃→CF₃CONH₂+C₂H₅OH
Formulation:
578.1 moles (82.1 kg) of ethyl trifluoroacetate
600.0 moles (10.2 kg) of ammonia
   Procedure:

The ethyl trifluoroacetate (82.1 kg) was introduced into the apparatus of Example 1, and ammonia was then introduced at room temperature. The reaction was exothermic, and the sump temperature increased to 86° C., depending on the rate of addition. While the ammonia was being introduced, the resulting alcohol and the ester were collected in the receiver and returned completely to the reactor during the reaction. The degree of conversion was monitored by GLC samples. At the end of the reaction, the ethyl alcohol was evaporated under a vacuum of 20 mbar at starting temperature of 55° C.

As the alcohol content decreased, the temperature was increased. The temperature at the head of the column was 18° to 19° C. The temperature in the sump and at the first length of pipe was maintained as high as possible, in order to be better able to remove the ethyl alcohol from the system. Refluxing was carried out at the rate of 3 liters per hour. Under vacuum (<20 mbar), the amide boiled at 82° C. The melt was boiled briefly in order to remove the last traces of ethanol. At the end of the reaction, the contents were cooled to 85° C., the equipment was vented, and the contents were pumped through the bottom outlet by the metering pump into the receiver of the cooling belt. The filling was carried out at static pressure in order to avoid pressure fluctuations. All the pipelines were heated.

The receiver and delivery unit or rotoformer were heated to 95° C. The amide was introduced at the rate of 14.4 kg per hour. The yield of trifluoroacetamide isolated in pellet form was 99% of the theoretical.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A method of preparing an organic compound which has a melting point higher than 30° C. at ambient pressure and is admixed with at least one organic contaminant having a boiling point of up to 120° C., said method comprising:
heating the mixture so that the mixture is maintained in a liquid state and the at least one organic contaminant is evaporated;
dividing the liquid from which the at least one organic contaminant has been evaporated into small portions;
and cooling the small portions to convert the portions into solid particles;
wherein the organic compound is thermally stable up to the boiling point thereof, and
wherein the boiling point of the organic compound is at least 40° C. higher than the boiling point of the at least one contaminant.

2. A method according to claim 1, wherein said organic compound is a peptide.

3. A method according to claim 2, wherein said peptide is substituted in the chain by fluorine.

4. A method according to claim 2, wherein said peptide has a fluorine-substituted protective group.

5. A method according to claim 1, wherein the organic compound is an acid amide corresponding to formula (I)
R—C(O)—NR¹R² (I)
wherein
R represents C1 to C4 alkyl, which is substituted by at least one fluorine atom, and
R¹ and R² are the same or different and each represent hydrogen or C1 to C4 alkyl;
or wherein
R¹ and R² together with the nitrogen atom form a five-membered or six-membered ring system;
said compound corresponding to formula (I) having a melting point of at least 40° at standard pressure; and
wherein said compound corresponding to formula (I) is prepared by reacting an ester of formula (II)
R—C(O)—OR³ (II)
wherein
R has the meaning given above, and
R³ represents C1 to C6 alkyl, substituted C1 to C6 alkyl, or aryl; with an amine corresponding to formula (III)
HNR¹ R² (III)
wherein
R¹ and R² have the meanings given above, in a reactor to obtain a mixture the acid amide corresponding to formula (I) and the alcohol R³OH, which is released from the ester corresponding to formula (III).
wherein the alcohol R³OH is distilled off as the contaminant while the contents of the reactor are kept in the liquid state, and the carboxylic acid amide corresponding to formula (I) is removed from the reactor as a liquid and divided into the smaller portions which are cooled to convert them to the solid particles.

6. A method according to claim 5, wherein R3 is phenyl or benzyl.

7. A method according to claim 5, wherein R represents CF₃, CF₂H, CF₂Cl, CF₃CFH or CF₃CF₂.

8. A method according to claim 5, wherein R¹ and R² are the same or different and each represent hydrogen, CH₃, C₂H₅ or C₃H₇.

9. A method according to claim 5, wherein R³ represents CH₃, C₂H₅, C₃H₇ or CF₃CH₂.

10. A method according to claim 5, wherein the small portions of liquid carboxylic acid amine are dripped onto a cooled support.

11. A method according to claim 10, wherein said cooled support comprises a cooled moving belt.

12. A method according to claim 5, wherein the liquid acid amide is divided into smaller portions by a rotating, perforated roller.

13. A method according to claim 5, wherein said smaller portions have a volume in the range from about 0.1 ml to about 20 ml.

14. An apparatus for producing a solid compound freed of a more volatile contaminant, said apparatus comprising:
a heated reactor
a rotatable perforated roller,
a pipeline connecting the heated reactor to the perforated roller, and
a cooled moving belt disposed to receive liquid droplets from said perforated roller.

15. An apparatus according to claim 14, wherein said pipeline is heated to maintain material conveyed therein in a liquid state.

16. An apparatus according to claim 14, further comprising a scraper for scraping solidified droplets from the cooled, moving belt.

17. An apparatus according to claim 14, further comprising a vacuum vessel on the pipeline between the heated reactor and the perforated for drawing off volatile contaminants.

18. An apparatus according to claim 14, wherein said reactor is provided with a stirrer.

19. An apparatus according to claim 14, wherein said moving belt is an endless metal belt.
